(19) United States
(12) Patent Application Publication
(10) Pub. No.: US 2026/0263507 A1
(43) Pub. Date: Sep. 10, 2026

(54) EYE DROP FORMULATIONS SUPPLEMENTED WITH CHONDROITIN SULFATE TO SOOTHE OPTICAL DISCOMFORT

(71) Applicant: Surface Ophthalmics, In., Pleasanton, CA (US)

(72) Inventors: Richard L. Lindstrom, Wayzata, MN (US); Dennis Saadeh, Brentwood, TN (US); Lyle Bowman, Pleasanton, CA (US)

(21) Appl. No.: 19/676,638

(22) Filed: May 13, 2026

Related U.S. Application Data
(63) Continuation-in-part of application No. 19/329,207, filed on Sep. 15, 2025.
Application No. 19/329,207 is a continuation-in-part of application No. 18/654,457, filed on May 3, 2024, now Pat. No. 12,440,510.
Application No. 18/654,457 is a continuation-in-part of application No. 18/555,982, filed on May 1, 2022, now Pat. No. 12,310,981.
Application No. 19/329,207 is a continuation-in-part of application No. PCT/US2025/024021, filed on Apr. 10, 2025.
Application No. PCT/US2025/024021 is a continuation of application No. 18/654,457, filed on May 3, 2024, now Pat. No. 12,440,510.
(60) Provisional application No. 63/186,747, filed on May 10, 2021.

(51) Int. Cl. A61K 31/737; A61K 9/00

(52) U.S. Cl. CPC A61K 31/737; A61K 9/0048

(57) ABSTRACT

Composition and its use for use treating optical redness and discomfort, the composition preferably having a viscosity about the same as water (e.g. 1-25 cps), an osmolality of 240-260 mOsm, and containing 0.1% to 2.5% wt/wt chondroitin sulfate, and an agent that reduces eye redness. Optical redness and discomfort is treated by topically administering a therapeutically effective amount of the composition to a subject's eye(s) suffering from the optical redness and discomfort.

### CROSS-REFERENCE TO RELATED APPLICATIONS

This application is a continuation in part of U.S. patent application Ser. No. 19/329,207, filed Sep. 15, 2025, which is a continuation in part of U.S. patent application Ser. No. 18/654,457, filed May 3, 2024, now U.S. Pat. No. 12,440,510, which is a continuation in part of U.S. patent application Ser. No. 18/555,982, filed Oct. 18, 2023, now U.S. Pat. No. 12,310,981, which is a US national phase application of PCT/US2022/027177, filed May 1, 2022, which claims priority to U.S. provisional patent application No. 63/186,747, filed May 10, 2021.

U.S. patent application Ser. No. 19/329,207 is also a continuation in part of international patent application no. PCT/US2025/024021, filed Apr. 10, 2025, which claims priority to U.S. patent application Ser. No. 18/654,457 filed 3 May 2024.

### TECHNICAL FIELD

The invention relates to eye drop formulations containing chondroitin sulfate and their use for treating ocular redness and discomfort.

### BACKGROUND OF THE INVENTION

Eye pain is generally characterized into two different categories: ocular pain, which occurs on the eye's surface; and orbital pain, which occurs within the eye. Ocular pain can be caused by allergy, conjunctivitis, irritation from contact lenses, corneal abrasion, burns, blepharitis, stye, dry eye disease, and other conditions.

In many ocular conditions, eye pain is also accompanied by ocular redness, which may arise from vasodilation and inflammation of conjunctival, episcleral, or other ocular surface blood vessels. For example, conditions such as dry eye disease, conjunctivitis, allergic irritation, corneal injury, blepharitis, and contact lens-associated irritation commonly present with both discomfort or pain and visible redness of the eye. Ocular redness may therefore serve as a clinical indicator associated with irritation, inflammation, or disruption of ocular surface homeostasis that contributes to or exacerbates ocular pain symptoms. In some patients, persistent redness may further correlate with burning, stinging, foreign body sensation, or general ocular discomfort, thereby negatively impacting visual comfort and quality of life.

Although various ophthalmic agents are known for reducing ocular redness, such treatments do not always adequately relieve the underlying ocular discomfort associated with irritation, dryness, inflammation, or pain. For example, various vasoconstrictive ophthalmic agents have been used to reduce redness of the eyes associated with irritation or inflammation by constricting superficial ocular blood vessels. Examples of such agents include naphazoline, phenylephrine, tetrahydrozoline, ephedrine, and brimonidine tartrate. These agents are generally administered to temporarily reduce the visible appearance of red eyes and improve cosmetic appearance. However, while such compounds may reduce redness, they often do not provide significant improvement in ocular comfort or relief from associated symptoms such as burning, stinging, dryness, irritation, or ocular pain. As a result, patients may continue to experience discomfort despite an observable reduction in ocular redness. Moreover, certain redness-reducing agents may themselves contribute to ocular discomfort, including transient stinging, irritation, or dryness following repeated use.

Thus there remains a need for improved treatments that also soothe discomfort associated with ocular disease and injury. In some instances, ocular discomfort may be caused or exacerbated by abnormalities in tear film osmolality associated with dry eye disease and other ocular surface pathologies. For example, elevated tear film osmolality, including tear hyperosmolarity, may contribute to ocular surface irritation, inflammation, and disruption of tear film homeostasis, resulting in symptoms such as dryness, burning, stinging, foreign body sensation, and ocular pain.

Chondroitin sulfate is one of the building blocks of cartilage and is believed to act as a protective coating for joints. In particular, it is believed that sulfate moieties of chondroitin sulfate interact with calcium ions in the bone to form a protective coating. WO 2006/058109 by Jafari et al. note that both chondroitin sulfate and glucosamine sulfate help build cartilage and can help delay the release of enzymes that breakdown cartilage. To this end, chondroitin sulfate has been used as a treatment to alleviate osteoarthritis pain and is often taken together with glucosamine sulfate.

In addition to its use in the treatment of osteoarthritis, WO 2006/058109 by Jafari et al. also describe a combination of chondroitin sulfate together with hyaluronic acid and glucosamine sulfate to form a triple polymer, viscoelastic agent to protect endothelial cells during cataract surgery. The term “viscoelastic” combines the words “viscous” and “elastic”. Viscous agents are resistant to flow, and elastic agents tend to return to their original shape after deformation. These features permit the Jafari et al. viscoelastic agent to be used during surgery to structurally maintain the corneal dome, maintain an anterior chamber depth (by filling the anterior chamber), and provide stability to the eye's anterior capsule surface. As such, Jafari et al. propose use of the triple polymer, viscoelastic agent at a surgical site during surgery, to protect the ocular tissue from trauma and therefore alleviate subsequent pain and inflammation associated with ocular surgery. Thus, while Jafari et al. disclose a viscoelastic agent to maintain optical structure during surgery to protect from trauma, Jafari et al. are silent as to the use of an eye drop formulation to treat redness and discomfort of the eyes after trauma has occurred.

U.S. Pat. No. 6,051,560 by Chang et al. found that adding chondroitin sulfate to sodium hyaluronate results in a synergistic viscosity due to hydrogen bonding between the molecules. Chang et al. disclose that administering this highly viscous composition before surgery protects corneal endothelial cells during intraocular lens (IOL) implantation, corneal transplantation, and other intraocular surgical operations. While Chang et al. provide a viscous solution for use before surgery, Chang et al. are silent as an eye drop formulation to treat redness and discomfort of the eyes after trauma has occurred.

U.S. Pat. No. 4,486,416 by Soll et al. also disclose the use of chondroitin sulfate prior to ocular surgery. In particular, it was found that chondroitin sulfate can eliminate the electrical attractive force between an intraocular lens and the endothelial layer of the cornea and thus, dipping an intraocular lens in a viscous solution of about 10-20% chondroitin sulfate prior to applying the lens to the eye, was found to reduce trauma. While the viscous solution disclosed in Chang et al. provide a protective coating prior to surgery, Chang et al. are silent as to an eye drop formulation to treat redness and discomfort of the eyes after the trauma has occurred.

U.S. Pat. No. 8,551,974 by Lindstrom found that a solution containing 2.5% chondroitin sulfate together with glycerol and dextran was an effective corneal pretreatment immediately before performing ophthalmic surgery. Consistent with prior uses of viscoelasitic agents, the composition formed a beneficial film over the cornea through which the surgery could be performed. While Lindstrom provides a film-like composition placed over the eye during surgery, Lindstrom is silent as to an eye drop formulation to treat redness and discomfort of the eyes after the trauma has occurred.

Accordingly, there remains a need for new treatments to relieve ocular redness and discomfort that can be self-administered by a subject suffering from ocular redness and discomfort, whether occurring after surgery or not in response to surgery.

### SUMMARY OF THE INVENTION

The invention is directed to compositions, preferably eye drop formulations, containing a low concentration of chondroitin sulfate together with an agent that reduces eye redness for the topical treatment of ocular redness and discomfort. In contrast to previous approaches of using chondroitin sulfate in highly viscous solutions to provide a protective coating to protect cells or tissue during surgery, the technical approach of the invention is to provide chondroitin sulfate at a low concentration, which contributes very little to the viscosity of the solution. It has been found that a low dose of chondroitin sulfate topically applied to the eye soothes the discomfort that often remains even when treating eye redness. Moreover, the eye drop formulation has a viscosity about the same as water and therefore minimizes adverse effects consistent with viscous solutions, such as blurred vision.

The above benefits have been surprisingly achieved in one aspect of the invention, by providing a composition for topical administration to a subject's eye, which includes chondroitin sulfate at a concentration of 0.1% to 2.5% wt/wt; an agent that reduces eye redness; and a pharmaceutically acceptable carrier. Preferably, the formulation is provided in an eye dropper bottle or eye dropper dose vial for topical application to the eye. This low concentration of chondroitin sulfate together with agent(s) for reducing redness in a low viscosity, hypo osmolar formulation was found surprisingly effective at relieving ocular discomfort in addition to redness. Moreover, the composition can be self-administered by the subject in need of eye redness and pain relief.

Nonlimiting examples of agents for reducing eye redness that can be combined with chondroitin sulflate include one or more of naphazoline, phenylephrine, tretrahydrozoline, epiphedrine, brimonidine tartrate, and pharmaceutically acceptable salt(s) thereof.

In various embodiments, the agent is naphazoline HCl at a concentration of 0.01% to 0.03% wt/wt; and/or phenylephrine HCl at a concentration of 0.08% to 0.20% wt/wt; and/or tretrahydrozoline HCl at a concentration of 0.01% to 0.05% wt/wt; and/or epiphedrine HCl at a concentration of 0.10% to 0.20% wt/wt; and/or brimonidine tartrate at a concentration of 0.02% to 0.05% wt/wt.

In some embodiments, the eye drop formulation also includes Dextran-70 and glycerol. In such embodiments, the concentrations of Dextran-70 and glycerol are such that they contribute little to the viscosity and osmolality of the eye drop formulation.

In preferred embodiments, the composition is free of hyaluronic acid and glucosamine sulfate.

While in some embodiments, the eye drop formulation contains a steroid and/or an immunosuppressant, preferably the eye drop formulation is steroid free and immunosuppressant free.

In a particularly preferred embodiment the eye drop formulation includes 0.1-2.5% wt/wt chondroitin sulfate, an agent that reduces eye redness (selected from one or more of 0.01%-0.03% wt/wt naphazoline HCl, 0.01%-0.05% wt/wt tretrahydrozoline HCl, 0.10%-0.20% wt/wt epiphedrine HCl, 0.02%-0.05% wt/wt brimonidine tartrate), 0.1%-1.0% wt/wt dextran-70, 0.03% wt/wt potassium chloride, 0.20% wt/wt PLURONIC F-127, 0.1-2% wt/wt glycerol, 0.6% wt/wt sodium phosphate dibasic anhydrous, 0.1% wt/wt sodium phosphate monobasic dihydrous, and water.

In another preferred embodiment the eye drop formulation includes 0.1-2.5% wt/wt chondroitin sulfate, an agent that reduces eye redness (e.g. 0.08%-0.20% wt/wt phenylephrine HCl) 0.1%-1.0% wt/wt dextran-70, 0.03% wt/wt potassium chloride, 0.20% wt/wt PLURONIC F-127, 0.1-2% wt/wt glycerol, 0.17% wt/wt citric acid, 0.06% wt/wt sodium citrate, and water.

In a related aspect of the invention, a composition for reducing eye redness and discomfort is provided, where the composition has a viscosity of 1 to 25 centipoise (cps) and an osmolality of 230-320 mOsm includes chondroitin sulfate at a concentration of 0.1% to 2.5% wt/wt; an agent that reduces eye redness; and a pharmaceutically acceptable carrier. The viscosity of the composition is preferably 1 to 12 cps. In some embodiments, the viscosity is 1 to 5 cps.

In some embodiments the composition has an osmolality of 230-320 mOsm. In further embodiments, the osmolality is 280-320 mOsm. In some embodiments the composition is an isotonic solution. The preferred osmolality is 240-260 mOsm.

In some embodiments, the composition also includes Dextran-70 and glycerol. In such embodiments, the concentrations of Dextran-70 and glycerol are such that they contribute little to the viscosity of the eye drop formulation so that the viscosity of the formulation remains between 1 to 25 cps, 1-12 cps, or 1-5 cps. In preferred embodiments the eye drop formulation is steroid free and free of immunosuppresants.

In another related aspect of the invention, a method for treating ocular redness and discomfort is also provided. In particular, the composition is topically administered to an eye of a subject suffering from redness and discomfort, preferably in the form of eye drops for self-administration. Among the benefits of the composition is that it significantly decreases eye redness and soothes the eyes to simultaneously improve appearance and comfort. This is accomplished in part by providing the eye drop formulation with a viscosity about the same as water or about 1-25 cps (more preferably 1-5 cps), which minimizes or avoids adverse effects associated with viscous solutions, such as blurred vision. Preferably, the eye drop formulations have a have a relatively low osmolality, such as 240-260 mOsm, which not only helps soothe ocular discomfort but can also support ocular surface health such as by helping to restore or support tear film homeostasis. However, in other embodiments the composition is an isotonic solution and thus has an osmolality of about 270-330 Osm. In still other embodiments, the composition has an osmolality from 230-320 mOsm.

In some embodiments, the ocular redness and discomfort results from receiving a medical treatment, such as ocular surgery. Eye drop formulations containing chondroitin sulfate at low concentration are demonstrated herein to be surprisingly effective at soothing the eyes after undergoing ocular surgery. Thus, in some embodiments, topical administration of the composition is used to treat redness and discomfort from an ocular surgery, which can be cataract surgery, refractive surgery or following intravitreal injections (IVI). In some embodiments, the composition containing chondroitin sulfate and agent that reduces eye redness is used to treat a subject suffering from surgical temporary ocular surgical discomfort syndrome (STODS).

In some embodiments the ocular redness and discomfort results from a medical condition, such as dry eye disease, contact lens induced ocular surface dry eye disease, preservative induced ocular surface disease, conjunctivitis, irritation from contact lenses, corneal abrasion, burns, blepharitis, or stye. Such uses include topically administering a therapeutically effective amount of the composition containing 0.1% to 2.5% wt/wt chondroitin sulfate with the agent that reduces redness to the subject's eye that is suffering from the ocular redness and discomfort resulting from the medical condition.

In some embodiments, the optical redness and discomfort is from an allergy, such as an eye allergy. In such instances, use can include topically administering a therapeutically effective amount of the composition containing 0.1% to 2.5% wt/wt chondroitin sulfate and the agent that reduces redness to the subject's eye that is suffering from the ocular redness and discomfort from the allergy.

In some embodiments, the optical redness and discomfort is from a corneal abrasion. In such instances, use can include topically administering a therapeutically effective amount of the composition containing 0.1% to 2.5% wt/wt chondroitin sulfate and the agent that reduces redness to the subject's eye that is suffering from the ocular redness and discomfort from the corneal abrasion.

In some embodiments, the optical redness and discomfort is from wearing a contact lens. In such instances, use can include topically administering a therapeutically effective amount of the composition containing 0.1% to 2.5% wt/wt chondroitin sulfate and the agent that reduces redness to the subject's eye that is suffering from the ocular redness and discomfort from wearing a contact lens.

In each of the embodiments, the compositions containing chondroitin sulfate are formulated to have a viscosity about the same as water (e.g. 1-25 cps but more preferably 1-5 cps) and are configured for administration topically to the eye.

In each of the embodiments, the composition are also preferably formulated to have an osmolality from 240 mOsm to about 260 mOsm. However, the compositions can also be provided in an isotonic solution (e.g. having an osmolality of about 270-330 mOsm). Still other compositions have an osmolality from 230-320 mOsm.

The compositions containing chondroitin sulfate and the agent(s) that reduces redness can be administered over any time sufficient to reduce ocular redness and discomfort. In instances of ocular surgery, the composition containing chondroitin sulfate and the agent that reduces redness can be administered over at least 7 days, optionally for 14 days or more after ocular surgery as a postop treatment regimen. In other instances, the composition containing chondroitin sulfate and the agent that reduces redness can be self-administered whenever the subject feels pain or discomfort or is displaying red eyes.

Chondroitin sulfate within the eye drop formulation can be at any suitable concentration from 0.1% to 2.5%, such as 0.1%, 0.15%, 0.2%, 0.25%, 0.3%, 0.35%, 0.4%, 0.45%, 0.5%, 0.55%, 0.6%, 0.65% 0.7%, 0.75% 0.8%, 0.85%, 0.9%, 0.95%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% wt/wt but the most preferred concentration is 0.1% to 0.25% wt/wt. Each of these concentrations of chondroitin sulfate contributes very little to the viscosity and osmolality of the eye drop formulation, thereby avoiding significant changes in viscosity/osmolality and minimizing instances of adverse effects such as blurred vision. Chondroitin sulfate can be derived from shark, bovine, or other sources.

### BRIEF DESCRIPTION OF DRAWINGS

FIG. 1 is a chart demonstrating percent pain clearance two weeks after cataract surgery, where the subjects received a topical eye drop formulation of either a combination of 0.2% wt/wt betamethasone sodium phosphate and 0.25% wt/wt chondroitin sulfate (left) or 0.25% wt/wt chondroitin sulfate (right).

### DETAILED DESCRIPTION OF THE INVENTION

As an introduction, the invention was surprisingly discovered while developing new formulations for the treatment of ocular pain. Among these included a formulation where chondroitin sulfate was added as part of the delivery vehicle, given its known acceptance in formulations used during surgery. However, unlike its conventional use during surgery, the concentration was significantly reduced, and the solution itself had a viscosity close to water. This low concentration was used at least in part because chondroitin sulfate is known to affect viscosity. Chondroitin sulfate is a thickening agent. However, among the design parameters of the new formulation was that it would have a low viscosity to 1) avoid overexposure to the active agent; 2) be configured as eye drops to be self-administered on the go by the subject; and 3) minimize blurred vision. Test formulations were developed to account for all three. Results during testing were surprising. Compositions containing chondroitin sulfate yielded unexpectedly high ocular pain relief in the tested patient populations.

Accordingly, the invention demonstrates a surprising reduction in ocular redness and discomfort when using eye drop formulations having a viscosity about the same as water and containing chondroitin sulfate at a low concentration together with an agent that reduces redness. That is, it was surprisingly found that the self-administration of eye drops containing a low dosage of chondroitin sulfate significantly soothes ocular pain and therefore significantly improves formulations that primarily target eye redness.

As used herein, the term “agent that reduces redness” refers to a compound, composition, or active pharmaceutical ingredient that, when administered to an eye or ocular tissue, decreases, suppresses, masks, or otherwise reduces the visible appearance of ocular redness or hyperemia. In certain embodiments, an agent that reduces redness may act, for example, through vasoconstriction of conjunctival or episcleral blood vessels, modulation of vascular tone, adrenergic receptor activity, or other mechanisms that reduce the appearance of redness.

Nonlimiting examples of agents that reduce redness include, without limitation, naphazoline, phenylephrine, tetrahydrozoline, ephedrine, brimonidine tartrate, pharmaceutically acceptable salts thereof, and combinations thereof.

In certain embodiments, the agent that reduces redness is present in an amount effective to reduce ocular hyperemia while maintaining acceptable tolerability and formulation stability. Nonlimited examples include naphazoline HCl at a concentration of about 0.01% to about 0.03% wt/wt, phenylephrine HCl at a concentration of about 0.08% to about 0.20% wt/wt, tetrahydrozoline HCl at a concentration of about 0.01% to about 0.05% wt/wt, ephedrine HCl at a concentration of about 0.10% to about 0.20% wt/wt, and/or brimonidine tartrate at a concentration of about 0.02% to about 0.05% wt/wt.

The term “agent that reduces redness” encompasses agents that reduce visible redness regardless of whether the agent also substantially improves ocular comfort, irritation, dryness, or pain.

As used herein, the term “discomfort” refers to any unpleasant ocular sensation, irritation, or subjective symptom experienced in or around the eye. Discomfort may include, without limitation, burning, stinging, itching, dryness, grittiness, foreign body sensation, soreness, aching, irritation, pressure, tenderness, watering, fatigue, or combinations thereof. In certain embodiments, discomfort may be associated with ocular redness, inflammation, dry eye disease, allergy, infection, contact lens wear, environmental exposure, surgical procedures, or other ocular conditions. The term “discomfort” encompasses symptoms of varying severity, including mild irritation through significant ocular pain, and may be transient, intermittent, or persistent.

As used herein, the term “soothe(s) discomfort” or “soothing discomfort” refers to reducing, calming, alleviating, relieving, lessening, or otherwise improving one or more symptoms of ocular discomfort experienced by a subject. In certain embodiments, soothing discomfort may include decreasing sensations such as burning, stinging, dryness, irritation, grittiness, foreign body sensation, soreness, aching, itching, tenderness, or ocular pain. The term encompasses both partial and complete relief of discomfort symptoms, whether temporary or sustained.

The ophthalmic compositions described herein can be formulated having any desired osmolality, such as between 230-320 mOsm. In some embodiments, the compositions are provided in an isotonic solution having an osmolality of about 270-330 mOsm. Preferably, the compositions have an osmolality of 240 mOsm to 260 mOsm. Without being bound by theory, it is believed that compositions having an osmolality range of 240-260 mOsm are most efficacious at sootheing ocular tissues and provide better ocular comfort when administered to eyes experiencing stinging, dryness, irritation, grittiness, foreign body sensation, soreness, aching, itching, tenderness, or ocular pain. For example, administration of a low-osmolality eye drop (e.g. 240-260 mOsm) may help counteract or reduce the effects of elevated tear film osmolality associated with dry eye disease and other ocular surface disorders. To this end, the low-osmolality carrier is preferably paired with the chondroitin sulfate and agent that reduces redness, such as a vasoconstrictive agent, thereby providing both reduction in visible ocular redness and still further improved ocular comfort. In certain instances, the low osmolality may reduce or lessen discomfort sometimes associated with redness-reducing agents, including burning, stinging, or dryness upon administration.

As introduced previously, chondroitin sulfate is a chemical normally found in cartilage around joints in the body. Commercially, it is both manufactured from animal sources (e.g. cow cartilage, shark cartilage) and produced chemically in the lab. Prior to cataract removal, chondroitin sulfate is typically injected into the eye to maintain a protective chamber during ocular surgery. While chondroitin sulfate is commonly used during surgical procedures in a viscous form, it is not conventionally prescribed during recovery. This disclosure family newly documents the benefits of using a low dose of chondroitin sulfate as a topical treatment for the treatment of already occurring ocular pain or discomfort. This disclosure family also newly documents the benefits of using chondroitin sulfate at low dose to treat pain or discomfort resulting from allergy, corneal abrasion, discomfort from wearing contact lenses, dry eye disease, ocular surgery induced dry eye disease, contact lens induced ocular surface dry eye disease, preservative induced ocular surface disease, surgical temporary ocular discomfort syndrome (STODS), and other ocular surface diseases and conditions that cause pain or discomfort in addition to eye redness. This disclosure family also documents an eye drop formulation having a viscosity about the same as water and containing chondroitin sulfate at a concentration from 1-5% wt/wt, more preferably 0.1-2.5% wt/wt, more preferably 0.1-1.0% wt/wt and most preferably 0.1-0.25% wt/wt. Still further, this disclosure family also documents eye drop formulations having an osmolality of 230-320 mOsm with a preferred osmolality of 240-260 mOsm.

To assess the safety, tolerability and efficacy of various solutions for treating ocular pain, a multicenter, randomized, double-masked, parallel group study was conducted. In particular, the study included approximately 182 subjects who underwent uncomplicated unilateral cataract surgery (phacoemulsification or extracapsular extraction; surgery without the aid of a femto laser). The subjects were treated with either a pharmaceutical composition of 0.2% wt/wt betamethasone sodium phosphate and 0.25% wt/wt chondroitin sulfate or a pharmaceutical composition of 0.25% wt/wt chondroitin sulfate. In particular, one drop of study drug was self-instilled by the subject (or instilled by the subject's caregiver) BID as directed (once in the morning and once in the evening, preferably 8-12 hours apart) in the surgery eye (study eye) for 16 days: the day prior to cataract surgery, the day of cataract surgery (at least one hour prior to surgery and then once in the evening following surgery), and continued BID postoperatively for 14 days.

The betamethasone/chondroitin sulfate pharmaceutical composition contained betamethasone sodium phosphate (0.2% betamethasone sodium phosphate) sodium thiosulfate, sodium 0.25% wt/wt chondroitin sulfate, Dextran-70, edetate disodium, poloxamer 407, glycerin, potassium chloride, sodium phosphate dibasic anhydrous, sodium phosphate monobasic anhydrous, sodium hydroxide, hydrochloric acid, and sterile water for injection. The osmolarity of this combination was between 240-260 mOsm. The formulation was sterile and non-preserved.

The chondroitin sulfate composition contained sodium thiosulfate, 0.25% wt/wt sodium chondroitin sulfate, Dextran-70, edetate disodium, poloxamer 407, glycerin, potassium chloride, sodium phosphate dibasic anhydrous, sodium phosphate monobasic anhydrous, sodium hydroxide, hydrochloric acid, and sterile water for injection. The osmolarity was between 240-260 mOsm. The formulation was sterile and non-preserved.

Primary efficacy analysis was conducted using a last-observation-carried-forward (LOCF) method. The primary efficacy endpoint is the proportion of subjects with an ACC grade of 0 at Day 15. Anterior chamber cells were counted and graded according to TABLE 1.

TABLE 1
Grading and Counting of
Anterior Chamber Cells

| Grade | Cell Count |
|---|---|
| 0 | 0 |
| 1 | 1-10 |
| 2 | 11-20 |
| 3 | 21-50 |
| 4 | >50 |

The secondary efficacy endpoint is the proportion of subjects who achieve a pain score of 0 on the VAS (0 to 100 mm scale) at each post-surgical assessment. Differences in proportions between the active treatment (with 0.2% betamethasone sodium phosphate) and placebo (without steroid) were calculated for each time point, with a 95% confidence interval for the differences. The secondary efficacy analysis is the proportion of subjects in each treatment group with an ACF grade of 0 at each study visit.

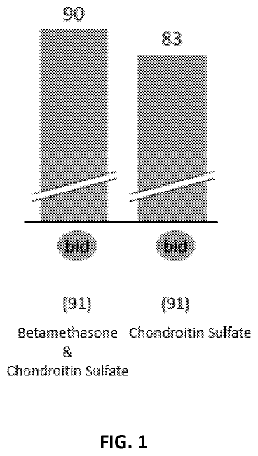

FIG. 1 is a chart of percent pain clearance at 2 weeks post-surgery. It was found that while the combined betamethasone/chondroitin sulfate composition was more efficacious than the chondroitin sulfate composition alone; the chondroitin sulfate composition, which is a steroid-free formulation performed surprisingly well even at low dose. The combined betamethasone/chondroitin sulfate treatment demonstrated 90% pain clearance and the chondroitin sulfate treatment demonstrated 83% pain clearance. This demonstrates the effectiveness of chondroitin sulfate to significantly soothe ocular pain when paired with an agent that reduces redness, thereby resulting in an eye drop formulation that both reduces redness and significantly soothes ocular pain or discomfort.

One having ordinary skill in the art to which the invention belongs would appreciate that the above demonstrates new and surprising therapeutic uses for chondroitin sulfate. In particular, chondroitin sulfate was highly efficacious at clearing ocular pain (83%) and can therefore supplement agents that primarily reduce eye redness. Moreover, the use of chondroitin sulfate will have fewer adverse treatment effects associated with other treatments. As such, the invention provides chondroitin sulfate itself as a complement to agents that reduce redness of the eyes to significantly improve the soothing of ocular pain or discomfort.

Still further, the chondroitin sulfate containing compositions described herein are preferably formulated for use as eye drops, which can be self-administered by the subject suffering from ocular redness and/or discomfort. Eye drop formulations for self-administration have materially different properties compared to conventional chondroitin sulfate containing solutions used before and during surgery. In particular, the chondroitin sulfate-based eye drops incorporate a low concentration of chondroitin sulfate and have a significantly lower viscosity compared to configurations used prior to and during surgery. In particular, the eye drop formulations have a viscosity about the same as water (e.g. 1-25 cps).

An implication of varying the viscosity of a delivery solution is that it can materially affect the therapeutic activity of the active agent and its adverse effects. On the one hand, formulations having a higher viscosity tend to stay longer on the eye surface, which can extend exposure to the therapeutic agent. On the other hand, a viscous formulation can disturb the subject's vision and can be more difficult to self-administer. Here, it was surprisingly found that a low dose of chondroitin sulfate was highly effective at treating ocular pain even at low viscosity, which provided further surprising benefits because its administration no longer required a viscous or gel-like form. The result is a chondroitin sulfate supplemented eye drop formulation having a viscosity close to water, which can therefore be self-administered without significant risk of visual disturbance.

The viscosity of preferred eye drop formulations disclosed herein are substantially lower than those conventionally used before and during surgery. This is accomplished at least in part by providing chondroitin sulfate at low concentrations (e.g. 0.1% to 2.5% wt/wt) since chondroitin sulfate is itself a known thickening agent. Moreover, maintaining a low viscosity can be achieved by choosing carriers and excipients that do not substantially affect the viscosity. As an example, hyaluronic acid and glucosamine sulfate, when used in combination with chondroitin sulfate, tend to thicken the formulation. As such, in preferred embodiments, the eye drop formulation is free of hyaluronic acid and glucosamine sulfate. However, compounds that affect the lubricious characteristics of the eye drop formulation but do not substantially affect the viscosity are preferred. As a nonlimiting example, a preferred eye drop formulation contains glycerol (e.g. 0.1%-2.0% wt/wt) and low concentration of Dextran-70 (e.g. 0.1% to 1%) to increase lubricity but at the same time, these amounts contribute minimally to the viscosity so it can remain close to that of water.

By having a viscosity “about the same as water” or “close to that of water,” it is meant that the viscosity of the formulation is from 1 cps or less, up to 25 cps at 20-25 degrees Celsius (“deg. C.”). In comparison, a gel is typically about 500-20,000 cps. More preferably, the viscosity of the formulation is from 1 cps to 11 cps, which is still closer to water. In some embodiments, the viscosity is less than three-fold the viscosity of water. In still further embodiments, the viscosity is from half the viscosity of water to two-fold the viscosity of water. As will be evident in the passages that follow, variability in viscosity measurement can be due to the variability of instrument settings and testing parameters, shear rate, etc, and is therefore better provided as a range rather than an absolute value.

As further demonstration, the viscosity of the formulation in Example 5 was measured using a Brookfield viscometer Model RVDII with a CP-41 spindle at 24 deg. C. using 2 mL of fluid at shear rates of 8 to 200 sec⁻¹, and a Rheometric Scientific SR5 rheometer with 40 mm cone and plate at 25.0 deg. C. at shear rates (sec-1) of 9.3 sec-1 to 477 sec⁻¹. The same formulation on the SR5 gave an average of viscosity of 2.323 cps as compared to water 2.333 cps. The Brookfield viscometer measured the same formulation at 1.32 cps compared to water at 2.16 cps. A Brookfield viscometer DVT-4 with a #0 spindle at 24 deg. C. gave a viscosity of 1.25 cps at a shear rate 60 sec⁻¹. Formulations were made with additional chondroitin sulfate up to approximately 10% and resulted in Newtonian viscosities of 5 to 11 cps with variations due to source/process of chondroitin sulfate raw material. Additionally, even though we see viscosity differences from 1 cps to 12 cps by sensitive measurements, this viscosity difference is indistinguishable by the human eye. In measuring the viscosity of these thixotropic formulations (20 secc⁻¹ to 40 sec⁻¹) or the Newtonian viscosity.

Although it should be clear that a viscosity close to that of water is preferred, one having ordinary skill in the art to which the invention belongs would recognize that viscosity can be increased, if desired, by adding thickening agent(s) until the desired viscosity is achieved. However, increasing the viscosity may also increase adverse effects, such as blurred vision. Nonetheless, nonlimiting examples of thickening agents to increase the viscosity of the eye drop formulations can include carboxymethylcellulose (CMC), hydroxypropyl methylcellulose (HPMC), hyaluronic acid (HA), glucosamine sulfate (GS) or others known in the pharmaceutical industry. That is, while the invention documents chondroitin sulfate at low dose and at low viscosity is surprisingly effective at treating optical pain and discomfort, chondroitin sulfate can also be used at higher dose and at a viscosity substantially greater than water. Thus, chondroitin sulfate can be used at 0.1-2.5% wt/wt, 2.5%-10%, wt/wt, or more and at viscosities of 1-25 cps, 25-50 cps, 50-100 cps, 100-1,000 cps, or more than 1,000 cps; however as viscosities increase it my be desirable to increase the size of a dropper aperture to account for the passage of thicker fluids or provide a different topical application technique such as a gell coated substrate for placement on the surface of the eye to be treated.

Thus, consideration of the viscosity should be taken into account when choosing the pharmaceutically acceptable carrier for delivering the chondroitin sulfate and agent that reduces redness.

It has also been surprisingly found that providing chondroitin sulfate in an eye drop formulation having an osmolality from about 240-260 mOsm provides improved soothing of the eyes. Without being bound by theory, it is believed that chondroitin sulfate may help protect, hydrate, or lubricate ocular surface tissues, while the low osmolality carrier may further assist in reducing ocular surface stress associated with ocular disease or injury. In certain embodiments, the topical delivery of chondroitin sulfate in a low osmolality carrier may provide enhanced soothing of symptoms such as burning, stinging, dryness, irritation, or foreign body sensation compared to formulations lacking chondroitin sulfate and/or lacking a low osmolality carrier. In some instances, the formulation may provide improved ocular comfort to counteract potential discomfort from an agent that reduces ocular redness. While preferred, the invention is not limited to carriers having low osmolality. The carrier could also be an isotonic solution, generally having an osmolality of about 270-320 mOsm, or an osmolality of 230-320 mOsm, but 240-260 mOsm is most preferred.

As used herein, the term “pharmaceutically acceptable carrier” refers to a vehicle, excipient, diluent, solvent, buffer, stabilizer, preservative, tonicity-adjusting agent, surfactant, lubricant, or other formulation component that is compatible with ocular administration and suitable for delivering one or more active agents to the eye without causing unacceptable toxicity, irritation, or adverse reaction under intended conditions of use. Preferably, the pharmaceutically acceptable carrier is formulated for topical ophthalmic administration as a low-viscosity, low osmolality, liquid composition, such as an ophthalmic solution or eye drop formulation having a viscosity approximating that of water and an osmolality of about 240-260 mOsm. Exemplary pharmaceutically acceptable carriers may include sterile aqueous solutions, saline solutions, buffered solutions, purified water, borate buffers, phosphate buffers, citrate buffers, sodium chloride, potassium chloride, glycerin, propylene glycol, polyethylene glycol, preservatives, and combinations thereof. Choosing between different buffers may be performed by one having ordinary skill in the art to which the invention belongs and may be determined in part by the agent for reducing redness. For example, it is currently more preferred to use a citrate buffer at acidic pH (e.g. 5.5-6.0) for phenylephrine, whereas using a phosphate buffer at a a pH closer to physiological pH (e.g. 7.0-7.4) for naphazoline may be preferred. Thus, it is preferred that the carrier is formulated to maintain suitable pH, osmolality (whether low, high or an isotonic solution), sterility, clarity, drop delivery characteristics, and ocular comfort while avoiding substantial increases in viscosity that could result in blurred vision, residue, or excessive coating of the ocular surface.

One having ordinary skill in the art to which the invention belongs will appreciate that the eye drop formulations containing 0.1% to 2.5% chondroitin sulfate and others can also contain, preservatives (though not preferred), electrolytes, and buffering agents as desired.

The eye drop formulations are preferably contained in a suitable container for use in administering eye drops. These include eye dropper bottles (e.g. droptainer) and eye dropper dose vials as known in the art to which the invention belongs. Preferably, the container is a preservative free container.

Turning now to the treatment of ocular pain or discomfort, the invention provides use of a composition, which itself is characterized by an eye drop formulation containing chondroitin sulfate at a concentration of 0.1% to 2.5% wt/wt and the agent that reduces redness in an eye dropper bottle or eye dropper dose vial, for treating ocular redness and discomfort, which includes topically administering to an eye of a subject suffering from ocular redness and discomfort, a therapeutically effective amount of the eye drop formulation containing 0.1 to 2.5% wt/wt chondroitin sulfate. Likewise, an exemplary method of treating ocular redness and discomfort can include topically administering to an eye of a subject suffering from a medical condition that results in ocular redness and discomfort, a therapeutically effective amount of the eye drop formulation containing 0.1% to 2.5% wt/wt chondroitin sulfate and the agent that reduces redness.

By “therapeutically effective amount” it is meant that the amount of administered chondroitin sulfate and agent that reduces redness is effective to alleviate or ameliorate ocular redness and discomfort. The therapeutically effective amount may also alleviate inflammation; stinging, burning or scratchy sensation in the eyes; stringy mucus in or around the eyes; sensitivity to light; eye redness; sensation of having something in the eyes; difficulty wearing contact lenses; difficulty with nighttime driving; watery eyes; blurred vision and eye fatigue. It is envisioned that a therapeutically effective amount may vary depending on the disorder or condition and its severity, the age, weight, etc. of the subject to be treated. A therapeutically effective amount can be provided all at once in a single administration or can be fractional amounts that provide the effect in several administrations. The precise determination of what would be considered therapeutically effective may be based on factors individual to each subject, including their size, age, injury and or severity of disease being treated and amount of time since the injury occurred or the disease began. As general guidance one or a few drops of an eye drop formulation containing 0.1% to 2.5% of chondroitin sulfate and the agent that reduces redness should be effective for most uses, and a 0.1% to 0.25% wt/wt chondroitin sulfate solution formulated as eye drops tends to be most preferred. Thus, chondroitin sulfate can be provided at 0.1%, 0.15%, 0.2%, 0.25%, 0.3%, 0.35%, 0.4%, 0.45%, 0.5%, 0.55%, 0.6%, 0.65% 0.7%, 0.75% 0.8%, 0.85%, 0.9%, 0.95%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% wt/wt; each of these eye drop formulations has a viscosity about the same as water (e.g. 1-25 cps). One having ordinary skill in the art to which the invention belongs will be able to determine the therapeutically effective amount based on these conditions which are routine in the art.

In view of the above, the 0.1% to 2.5% chondroitin sulfate together with the agent that reduces redness can be provided in an acceptable or suitable carrier, such as a diluent, buffer, or excipient, that is compatible with the chondroitin sulfate, agent, and any other ingredients forming the eye drop formulation and is suitable for topical administration to the eye as long as the viscosity remains about the same as water (e.g. 1-25 cps) and at the desired osmolality range. A nonlimiting example is the formulation of eye drops for topical administration having chondroitin sulfate at a concentration of 0.1% to 2.5% wt/wt, or at a concentration of 0.1% to 1.0%, or 0.1% to 0.25% wt/wt. Thus, chondroitin sulfate can be provided at a concentration of 0.1%, 0.15%, 0.2%, 0.25%, 0.3%, 0.35%, 0.4%, 0.45%, 0.5%, 0.55%, 0.6%, 0.65% 0.7%, 0.75% 0.8%, 0.85%, 0.9%, 0.95%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% wt/wt; each of these eye drop formulations has a viscosity about the same as water (e.g. 1-25 cps) and does not substantially affect osmolality. When adding additional agents, the viscosity should be substantially maintained to remain about the same as water and preferably osmolality should remain about 240-260 mOsm but an isotonic osmolality could also be used or any between about 230-320 mOsm. For instance small amounts of dextran-70 (e.g. 0.1-1% wt/wt) and glycerol (e.g. 0.1-2% wt/wt) can be added.

As demonstrated herein, topical administration of eye drops containing chondroitin sulfate and the agent that reduces redness significantly relieves ocular redness and discomfort after undergoing ocular surgery and is therefore demonstrated useful for treating various surgery related conditions such as surgical temporary ocular discomfort syndrome (STODS). STODS can occur following any ocular surgery or procedure that disrupts the ocular surface, such as refractive surgery, cataract surgery, and intravitreal injections (IVI). Without being bound by theory, it is believed that the chondroitin sulfate supplemented eye drop formulation reduces cellular oxidated stress and edema to achieve cell membrane stabilization, enhances ocular surface lubrication and prolongation of contact time, and preserves ocular surface homeostasis by proactively addressing tear film hyperosmolarity.

The skilled artisan will appreciate that the eye drop formulations containing chondroitin sulfate and the agent that reduces redness can also be used to treat dry eye disease more broadly. Dry eye disease is frequently caused by decreased tear production. Decreased tear production can occur as a result of age, hormonal changes, various autoimmune diseases, and other factors, and may also be a side effect of certain medications, such as beta-blockers, antidepressants, antihistamines, etc.

Dry eye disease can also occur after ocular surgery. Manipulation and contact between instruments, medications, surgical tools, and the cornea can cause microscopic damage to the eye, thereby resulting in temporary disturbance to the ocular surface, which if left untreated can develop into a chronic condition. For patients with pre-existing dry eye, ocular surgery can exacerbate existing symptoms.

Eye drop formulations containing 0.1% to 2.5% wt/wt chondroitin sulfate and the agent that reduces redness can also be used to treat contact lens discomfort or contact lens induced ocular surface dry eye disease. Contact lens discomfort is often associated with dry eyes. A contact lens divides the tear film into two layers: the pre and post-lens tear film. Bifurcating the film into two layers increases friction between the contact lens and ocular surface, which can cause dry eye disease. Accordingly, also provided are 1) a method of treating ocular redness and discomfort from contact lens wear, which includes topically administering a therapeutically effective amount of an eye drop formulation containing 0.1% to 2.5% wt/wt chondroitin sulfate and the agent that reduces redness to an eye of a subject suffering from redness and discomfort from wearing contact lenses; and 2) a method of treating an ocular surface disease, which includes topically administering a therapeutically effective amount of an eye drop formulation containing 0.1% to 2.5% wt/wt chondroitin sulfate and the agent that reduces redness to an eye of a subject suffering from an ocular surface disease. In furtherance of this, in some embodiments ocular surface disease is a dry eye disease, such as contact lens induced ocular surface dry eye disease. In each, preferably the eye drop formulation has a viscosity that is about the same as water (e.g. 1-25 cps) and an osmolality of about 240-260 mOsm, although an isotonic solution could also be used.

The eye drop formulations containing 0.1% to 2.5% wt/wt chondroitin sulfate and the agent that reduces redness can also be used to treat other ocular surface diseases. For example, glaucoma medications can be associated with toxicities to the ocular surface. Common therapies for glaucoma treatment include the use of prostaglandin analogs, beta-adrenergic antagonists, alpha-adrenergic agonists, and carbonic anhydrase inhibitors. Due to either the added preservative or the active ingredient of the medication itself, these topical treatments for glaucoma can cause or worsen ocular surface disease. Moreover, preservatives such as benzalkonium chlorides have widespread applications, including use in eye drops. Such preservatives can cause dry eye disease and are therefore preferably absent from the eye drop formulations. Accordingly, an eye drop formulation containing 0.1% to 2.5% wt/wt chondroitin sulfate and the agent that reduces redness can be used to treat ocular surface disease induced by preservatives or other active ingredients, such as those used in the treatment of glaucoma. As such, methods for treating ocular surface disease can include topically administering a therapeutically effective amount of an eye drop formulation containing 0.1% to 2.5% wt/wt chondroitin sulfate and the agent that reduces redness to an eye of a subject suffering from preservative induced ocular surface disease; and another exemplary embodiment includes topically administering a therapeutically effective amount of an eye drop formulation containing 0.1% to 2.5% wt/wt chondroitin sulfate and the agent that reduces redness to an eye of a subject suffering from glaucoma. Preferably, the viscosity is about the same as water to avoid blurred vision or other adverse effects associated with viscous solutions.

Eye drop formulations containing 0.1% to 2.5% wt/wt chondroitin sulfate and the agent that reduces redness can also be used to treat eye pain or discomfort from allergies, such as eye allergies. Eye allergy pain is caused by an inflammatory response triggered when the eyes come into contact with allergens like pollen, dust mites, pet dander, mold, or smoke. Inflammation and irritation can cause blood vessel dilation (leading to redness), increased tear production, swelling (edema) of tissue, and/or stimulation of nerve endings resulting in pain, itching or burning sensations. Treatment of eye redness and discomfort from eye allergy can include topically administering to an eye of the subject suffering from the eye pain a therapeutically acceptable amount of an eye drop formulation containing 0.1% to 2.5% wt/wt chondroitin sulfate and the agent that reduces redness. In each of these, the viscosity is about the same as water (e.g. 1-25 cps). Without being bound by theory it is believed that chondroitin sulfate reduces edema and itchiness when used to treat allergy.

Though nonlimiting, this disclosure documents that topical administration of chondroitin sulfate at low dose, twice daily (BID) for two weeks, from the day of receiving cataract surgery, cleared 83% of ocular pain. As such, topical administration of eye drops containing chondroitin sulfate together with the agent that reduces redness for less than two weeks after receiving ocular surgery will also clear pain in addition to redness; however, pain clearance might be below 83% in some subjects. Accordingly, within the scope of the disclosure, an eye drop formulation containing chondroitin sulfate and the agent that reduces redness is preferably topically administered for at least 1 day, 2 days, 3 days, 4 days, 5 days, 6 days, 7 days, 8 days, 9 days, 10 days, 11 days, 13 days, 14 days or more. While chondroitin sulfate can soothe pain or discomfort within the first day, it has also been found that long term use is also effective. In some instances, eye drops containing chondroitin sulfate can be administered when the subject feels pain or discomfort. The artisan will also appreciate that while chondroitin sulfate and the agent that reduces redness is preferably administered topically via eye drops twice daily (BID), it can also be administered once daily, three times daily (TID), four times daily (QID) or more.

While the eye drop formulations containing chondroitin sulfate have been found highly efficacious at treating ocular pain, it is beneficial to combine the chondroitin sulfate with the agent that reduces redness, especially in instances of significant inflammation or redness. For example, significant inflammation may occur after ocular surgery and thus a subject that has undergone ocular surgery may be provided postop with a pharmaceutical composition including chondroitin sulfate and the agent that reduces redness. In such compositions, the active ingredients (e.g. chondroitin sulfate and the agent that reduces redness) are provided in a therapeutically effective amount. In furtherance of the above, the chondroitin sulfate was also administered topically to the eye with the corticosteroid betamethasone sodium phosphate during clinical testing. This combined betamethasone/chondroitin sulfate composition was administered twice daily (BID) for two weeks, from the day of receiving cataract surgery. The combined betamethasone/chondroitin sulfate composition cleared 90% of pain and 56% of inflammation. As such, the addition of an anti-inflammatory agent is also encompassed by the methods and compositions described within this document.

### EXAMPLES

### Example 1

### Preparing Eye Drop Formulation No. 1

Eye drop formulations were prepared as described below. The following products were used in the amounts specified in TABLE 2:

TABLE 2
Pharmaceutical Composition No. 1

| Amount | Product |
|---|---|
| 0.25 g | chondroitin sulfate (bovine) |
| 0.25 g | powdered dextran-70 |
| 0.1 g | edetate disodium powder |
| 0.03 g | potassium chloride |
| 0.20 g | PLURONIC ^{®} F-127 |
| 0.2 g | glycerol |
| 0.9 g | sodium phosphate dibasic anhydrous |
| 0.18 g | sodium phosphate monobasic anhydrous |
| 100 mL | sterile injectable water |

Chondroitin sulfate, dextran, potassium phosphate, dibasic and monobasic sodium phosphate, potassium chloride, EDTA, and PLURONIC® F-127 were combined with about 90% of water and stirred until completely dissolved followed by adding glycerol with continued stirring. The pH of the solution was then adjusted to about 7.0-7.4 using sodium hydroxide solution. The solution was then filtered through a 0.2 micron filter into a sterile droptainer and sealed for subsequent topical delivery to the eye as eye drops. The viscosity of this solution was about the same as water, and the osmolality was between 240-260 mOsm.

### Example 2

### Preparing Eye Drop Formulation No. 2

Eye drop formulations were prepared as described below. The following products were used in the amounts specified in TABLE 3:

TABLE 3
Pharmaceutical Composition No. 2

| Amount | Product |
|---|---|
| 0.50 g | chondroitin sulfate (bovine) |
| 0.25 g | powdered dextran-70 |
| 0.1 g | edetate disodium powder |
| 0.03 g | potassium chloride |
| 0.20 g | PLURONIC ^{®} F-127 |
| 0.2 g | Glycerol |
| 0.9 g | sodium phosphate dibasic anhydrous |
| 0.18 g | sodium phosphate monobasic anhydrous |
| 100 mL | sterile injectable water |

Chondroitin sulfate, dextran, potassium phosphate, dibasic and monobasic sodium phosphate, potassium chloride, EDTA, and PLURONIC® F-127 were combined with about 90% of water and stirred until completely dissolved, followed by adding glycerol with continued stirring. The pH of the solution was then adjusted to about 7.0-7.4 using sodium hydroxide solution. The solution was then filtered through a 0.2 micron filter into a sterile droptainer and sealed for subsequent topical delivery to the eye as eye drops. The viscosity of this solution was about the same as water, and the osmolality was between 240-260 mOsm.

### Example 3

### Preparing Eye Drop Formulation Nos. 3 and 4

An eye drop formulation was prepared as described below. The following products were used in the amounts specified in TABLE 4:

TABLE 4
Preparation of Pharmaceutical Composition Nos. 3 and 4

| Amount | Product |
|---|---|
| 0.02 or 0.04 g | betamethasone sodium phosphate |
| 0.25 g | chondroitin sulfate (bovine) |
| 0.25 g | powdered dextran-70 |
| 0.1 g | edetate disodium powder |
| 0.10 g | powdered sodium thiosulfate pentahydrate |
| 0.03 g | potassium chloride |
| 0.20 g | PLURONIC ^{®} F-127 |
| 0.2 g | glycerol |
| 0.9 g | sodium phosphate dibasic anhydrous |
| 0.18 g | sodium phosphate monobasic anhydrous |
| 100 mL | sterile injectable water |

Chondroitin sulfate, dextran, sodium thiosulfate, phosphate buffer, potassium chloride, EDTA, and PLURONIC® F-127 were combined with about 90% of the water and stirred until completely dissolved followed by addition of glycerol with continued stirring. The pH of the solution was then adjusted to about 7.0 using sodium hydroxide. A stock solution of 1% betamethasone sodium phosphate was prepared by dissolving 1 gm of betamethasone sodium phosphate powder in 100 ml water and confirming the stock solution concentration by HPLC. Then 2 (Formulation 3) or 4 ml (Formulation 4) of the 1% betamethasone sodium phosphate stock solution was then added to the mixture with continued stirring. The solution was then filtered through a 0.2 micron filter into sterile dropper bottles or unit dose vials for subsequent topical delivery to the eye as eye drops. The viscosities of these solutions are about the same as water, and the osmolalities were between 240-260 mOsm.

### Example 4

### Preparing Eye Drop Formulation No. 5

An eye drop formulation was prepared as described below. The following products were used in the amounts specified in TABLE 5:

TABLE 5
Preparation of Pharmaceutical Composition No. 5

| Amount | Product |
|---|---|
| 0.2 g | betamethasone sodium phosphate |
| 0.25 g | chondroitin sulfate (bovine) |
| 0.25 g | powdered dextran-70 |
| 0.1 g | edetate disodium powder |
| 0.10 g | powdered sodium thiosulfate pentahydrate |
| 0.015 g | potassium chloride |
| 0.20 g | PLURONIC ^{®} F-127 |
| 0.2 g | Glycerol |
| 0.95 g | sodium phosphate dibasic anhydrous |
| 0.2 g | sodium phosphate monobasic anhydrous |
| 100 mL | sterile injectable water |

Chondroitin sulfate, dextran, sodium thiosulfate, phosphate buffer, potassium chloride, EDTA, and PLURONIC® F-127 were combined with about 90% of the water and stirred until completely dissolved followed by addition of glycerol with continued stirring. The pH of the solution was then adjusted to about 7.0 using sodium hydroxide. A stock solution of 1% betamethasone sodium phosphate was prepared by dissolving 1 gm of betamethasone sodium phosphate powder in 100 ml water and confirming the stock solution concentration by HPLC. Then 20 ml of the 1% betamethasone sodium phosphate stock solution was then added to the mixture with continued stirring. The solution was then filtered through a 0.2 micron filter into sterile dropper bottles or unit dose vials for subsequent topical delivery to the eye as eye drops. The viscosity of this solution was about the same as water, and the osmolality was between 240-260 mOsm.

### Example 5

### Preparing Eye Drop Formulation No. 6

An eye drop formulation was prepared as described below. The following products were used in the amounts specified in TABLE 6:

TABLE 6
Preparation of Pharmaceutical Composition No. 6

| Amount | Product |
|---|---|
| 0.1 g | chondroitin sulfate (bovine) |
| 0.1 g | powdered dextran-70 |
| 0.003 g | potassium chloride |
| 0.20 g | PLURONIC ^{®} F-127 |
| 1.00 g | Glycerol |
| 0.6 g | sodium phosphate dibasic anhydrous |
| 0.1 g | sodium phosphate monobasic anhydrous |
| 100 mL | sterile injectable water |

Chondroitin sulfate, dextran, phosphate buffer, potassium chloride, and, PLURONIC® F-127 were combined with about 90% of the water and stirred until completely dissolved followed by addition of glycerol with continued stirring. The pH of the solution was then adjusted to about 7.0 using sodium hydroxide. The solution was then filtered through a 0.2 micron filter into sterile dropper bottles or unit dose vials for subsequent topical delivery to the eye as eye drops. The viscosity of this solution was about the same as water, and the osmolality was between 240-260 mOsm.

### Examples 6-10

### Preparing Eye Drop Formulation Nos. 7-10

Eye drop formulations are prepared as described below. The following products are used in the amounts specified in TABLE 7 and as described below.

TABLE 7
Preparation of Eye Drop Formulation Nos. 7-10

| Amount | | Product |
|---|---|---|
| 0.05-2.5 | g | chondroitin sulfate (bovine) |
| various | | agent that reduces eye redness |
| 0.1 | g | powdered dextran-70 |
| 0.03 | g | potassium chloride |
| 0.20 | g | PLURONIC ^{®} F-127 |
| 1.00 | g | Glycerol |
| Just 0.6 | g | sodium phosphate dibasic anhydrous |
| 0.1 | g | sodium phosphate monobasic anhydrous |
| 100 | mL | sterile injectable water |

The “agent that reduces eye redness” in the above formulations are: formulation 7:0.01-0.03 g naphazoline HCl; formulation 8:0.01-0.05 g tretrahydrozoline HCl; formulation 9:0.01-0.20 g epiphedrine HCl; and formulation 10:0.02-0.05 g brimonidine tartrate.

Chondroitin sulfate, the corresponding agent that reduces eye redness, dextran, potassium phosphate, dibasic and monobasic sodium phosphate, potassium chloride, EDTA, and PLURONIC® F-127 are combined with about 90% of water and stirred until completely dissolved, followed by adding glycerol with continued stirring. The pH of the solution is then adjusted to about 7.0-7.4 using sodium hydroxide solution. The solution is then filtered through a 0.2 micron filter into a sterile, preservative free bottle, then sealed for subsequent topical delivery to the eye as eye drops. The viscosities of these solutions are about the same as water, and the osmolalities are between 240-260 mOsm.

### Example 11

### Preparing Eye Drop Formulation No. 11

An eye drop formulation is prepared as described below. The following products are used in the amounts specified in TABLE 8.

TABLE 8
Preparation of Eye Drop Formulation No. 12

| Amount | | Product |
|---|---|---|
| 0.1 | g | chondroitin sulfate (bovine) |
| 0.01-0.03 | g | naphazoline HCI |
| 0.1 | g | powdered dextran-70 |
| 0.03 | g | potassium chloride |
| 0.20 | g | PLURONIC ^{®} F-127 |
| 1.00 | g | Glycerol |
| 0.6 | g | sodium phosphate dibasic anhydrous |
| 0.1 | g | sodium phosphate monobasic anhydrous |
| 100 | mL | sterile injectable water |

Chondroitin sulfate, naphazoline HCl, dextran, potassium phosphate, dibasic and monobasic sodium phosphate, potassium chloride, EDTA, and PLURONIC® F-127 are combined with about 90% of water and stirred until completely dissolved, followed by adding glycerol with continued stirring. The pH of the solution is then adjusted to about 7.0-7.4 using sodium hydroxide solution. The solution is then filtered through a 0.2 micron filter into a sterile droptainer or other suitable preservative free bottle and sealed for subsequent topical delivery to the eye as eye drops. The viscosity of this solution is about the same as water, and the osmolality is between 240-260 mOsm.

### Example 12

### Preparing Eye Drop Formulation No. 12

An eye drop formulation is prepared as described below. The following products are used in the amounts specified in TABLE 9.

TABLE 9
Preparation of Eye Drop Formulation No. 13

| Amount | | Product |
|---|---|---|
| 0.1 | g | chondroitin sulfate (bovine) |
| 0.08-0.20 | g | phenylephrine HCl |
| 0.1 | g | powdered dextran-70 |
| 0.03 | g | potassium chloride |
| 0.20 | g | PLURONIC ^{®} F-127 |
| 1.00 | g | Glycerol |
| 0.17 | g | Citric acid |
| 0.06 | g | sodium citrate |
| 100 | mL | sterile injectable water |

Chondroitin sulfate, phenylephrine HCl, dextran, citric acid, sodium citrate, potassium chloride, EDTA, and PLURONIC® F-127 are combined with about 90% of water and stirred until completely dissolved, followed by adding glycerol with continued stirring. The pH of the solution is then adjusted to about 5.5-6.0. The solution is then filtered through a 0.2 micron filter into a sterile droptainer or other suitable preservative free bottle and sealed for subsequent topical delivery to the eye as eye drops. The viscosity of this solution is about the same as water, and the osmolality is between 240-260 mOsm.

Although the invention has been described with the reference to the above examples, it will be understood that modifications and variations are encompassed within the spirit and scope of the invention. Accordingly, the invention is limited only by the finally granted claims.

What is claimed is:

1. A composition for topical administration to a subject's eye, the composition comprising:
chondroitin sulfate or a pharmaceutically acceptable salt thereof at a concentration of 0.05% to 2.5% wt/wt;
an agent that reduces eye redness; and
a pharmaceutically acceptable carrier.

2. The composition of claim 1, wherein the agent for reducing eye redness is one or more agent selected from the group consisting of naphazoline, phenylephrine, tretrahydrozoline, epiphedrine, brimonidine tartrate, and pharmaceutically acceptable salt(s) thereof.

3. The composition of claim 1, wherein the agent is naphazoline HCl at a concentration of 0.01% to 0.03% wt/wt.

4. The composition of claim 1, wherein the agent is phenylephrine HCl at a concentration of 0.08% to 0.20% wt/wt.

5. The composition of claim 1, wherein the agent is tretrahydrozoline HCl at a concentration of 0.01% to 0.05% wt/wt.

6. The composition of claim 1, wherein the agent is epiphedrine HCl at a concentration of 0.10% to 0.20% wt/wt.

7. The composition of claim 1, wherein the agent is brimonidine tartrate at a concentration of 0.02% to 0.05% wt/wt.

8. The composition of claim 1, further comprising a dextran and a glycerin.

9. The composition of claim 8, wherein the dextran is dextran-70 and is at a concentration of 0.05% to 0.3% wt/wt, optionally 0.10% wt/wt, and the glycerin is at a concentration of 0.50% to 3%, wt/wt, optionally 1% wt/wt.

10. The composition of claim 1, comprising a viscosity of 1-25 centipoise.

11. The composition of claim 1, comprising an osmolality of 240-260 mOsm.

12. The composition of claim 1 in a dispenser suitable for topical application of the composition to the subject's eye.

13. A method of reducing eye redness and discomfort in a subject, the method comprising topically administering the composition of claim 1 to a subject's eye that is suffering from redness and discomfort.

14. The method of claim 13, wherein the subject is suffering from an ocular surface disease or ocular injury.

15. The method of claim 13, wherein the subject is suffering from one or more condition selected from the group consisting of dry eye, an eye allergy, a corneal abrasion, and surgical temporary ocular discomfort syndrome (STODS).

16. The method of claim 13, wherein the eye redness and discomfort are in response to contact lens wear or ocular surgery.

17. The method of claim 13, wherein the discomfort is ocular pain.

18. The method of claim 13, wherein the discomfort is characterized by one or more sensation selected from the group consisting of burning, stinging, itching, dryness, grittiness, foreign body sensation, soreness, aching, pressure, and tenderness.
